(19) United States
(12) Patent Application Publication
(10) Pub. No.: US 2024/0033117 A1
(43) Pub. Date: Feb. 1, 2024

(54) A COLLECTION DEVICE

(71) Applicant: Salts Healthcare Limited, Birmingham (GB)

(72) Inventors: Marcus Allen, Birmingham (GB); Mark Heynes, Beaconside (GB); John Harverson, Beaconside (GB); Phillip Sampey, Beaconside (GB)

(21) Appl. No.: 18/275,582

(22) PCT Filed: Feb. 3, 2022
(86) PCT No.: PCT/GB2022/050286

§ 371 Date: Aug. 2, 2023

(30) Foreign Application Priority Data
Feb. 5, 2021 (GB) 2101651.4

(51) Int. Cl. A61F 5/44; A61F 5/441; B65B 69/00

(52) U.S. Cl. CPC A61F 5/4404; A61F 5/441; B65B 69/0016

(57) ABSTRACT

An ostomy collection device (10) including: a housing (12) which defines a collecting volume for receiving and storing waste from an ostomy appliance, an inlet (30) for fluidly connecting to an outlet of an ostomy appliance so that waste from the ostomy appliance flows into the collecting volume, an outlet (32) for emptying the contents of the collecting volume, and a lid portion (40) which is pivotable between a first position, in which the outlet (32) is open and waste can flow through the outlet (32) from the collecting volume, and a second position in which waste flow through the outlet (32) is impeded or substantially prevented.

### DESCRIPTION OF INVENTION

Embodiments of the present invention relate to an ostomy collection device.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

In such times, it would be beneficial to provide an additional collecting volume that is connected to the existing ostomy appliance, so that the appliance does not need to be emptied as often. This functionality is currently provided by a flexible bag which is connected to the ostomy appliance.

Alternative systems are sought for ostomy appliance users. Embodiments of the present invention seeks to alleviate one or more drawbacks of the prior art.

According to an aspect of the invention, we provide an ostomy collection device including: a housing which defines a collecting volume for receiving and storing waste from an ostomy appliance, an inlet for fluidly connecting to an outlet of an ostomy appliance so that waste from the ostomy appliance flows into the collecting volume, an outlet for emptying the contents of the collecting volume, and a lid portion which is pivotable between a first position, in which the outlet is open and waste can flow through the outlet from the collecting volume, and a second position in which waste flow through the outlet is impeded.

Further optional features are set out in the appended claims.

Embodiments of the present invention are described, by way of example only, with reference to the accompanying drawings, in which:

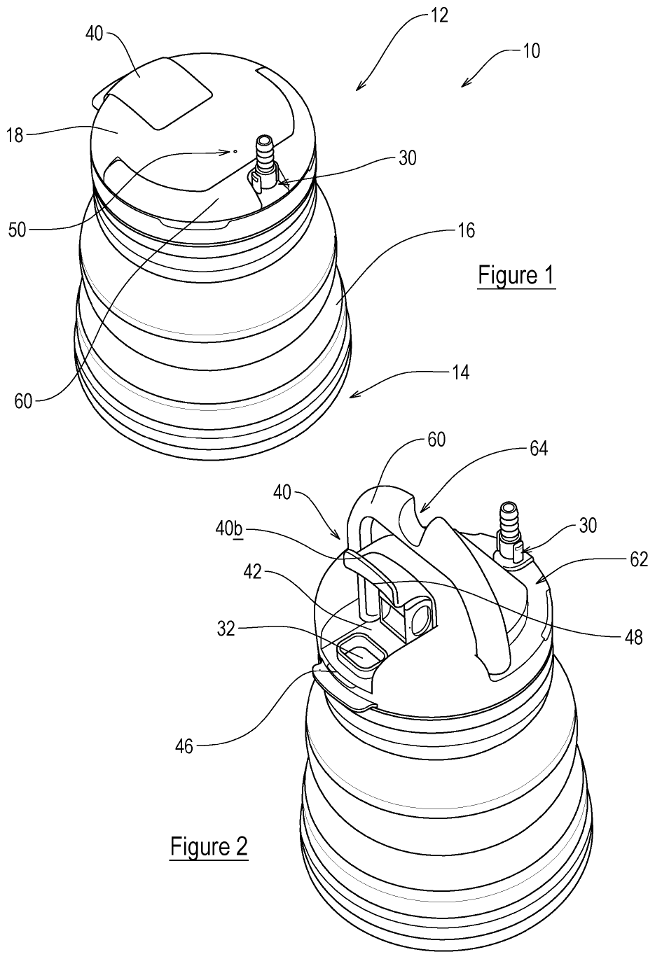

FIG. 1 is perspective views of an ostomy collection device,

FIG. 2 is perspective view of the ostomy collection device,

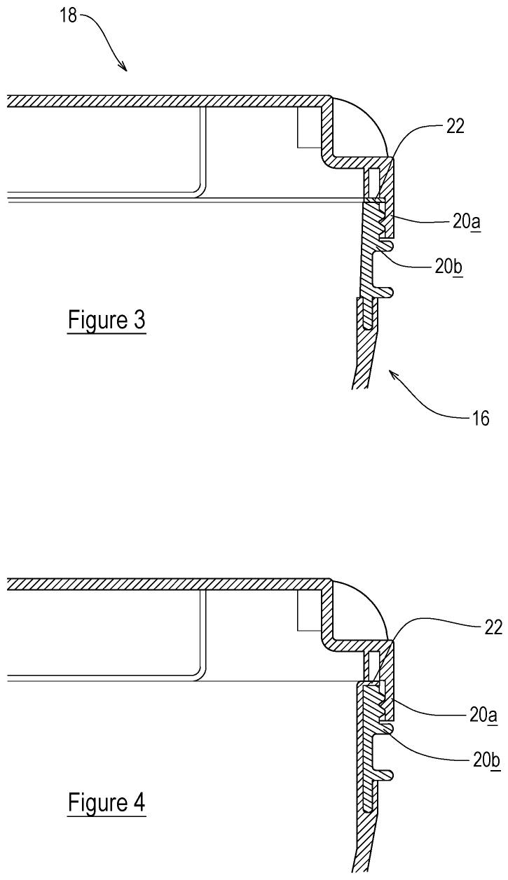

FIG. 3 is a cross-sectional view of part of an ostomy collection device, and

FIG. 4 is a cross-sectional view of a part of another embodiment

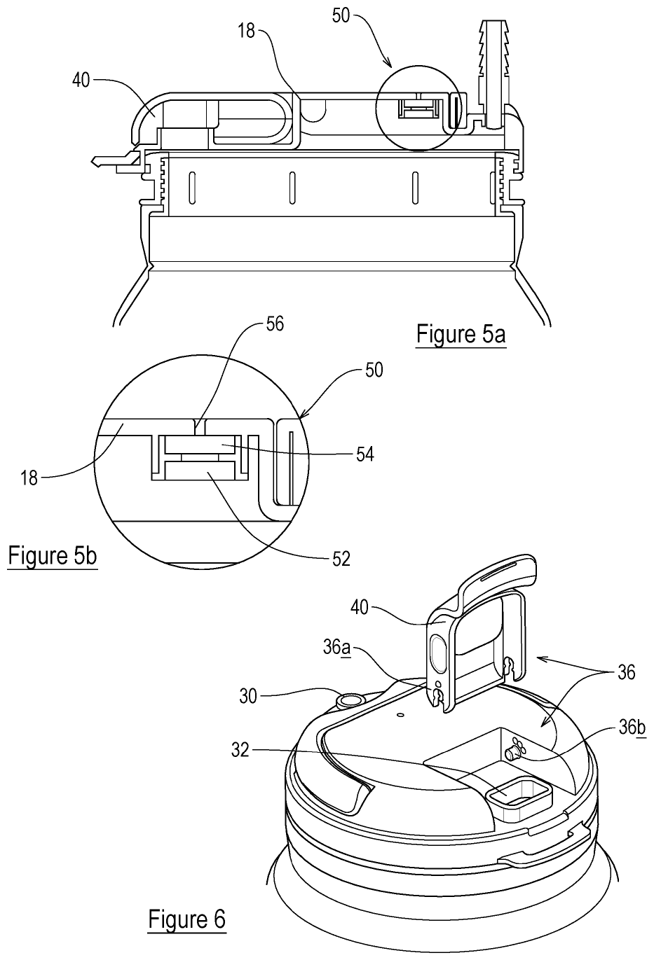

FIGS. 5a and 5b illustrate an air vent opening of an ostomy collection device,

FIG. 6 is a perspective view of part of the ostomy collection device,

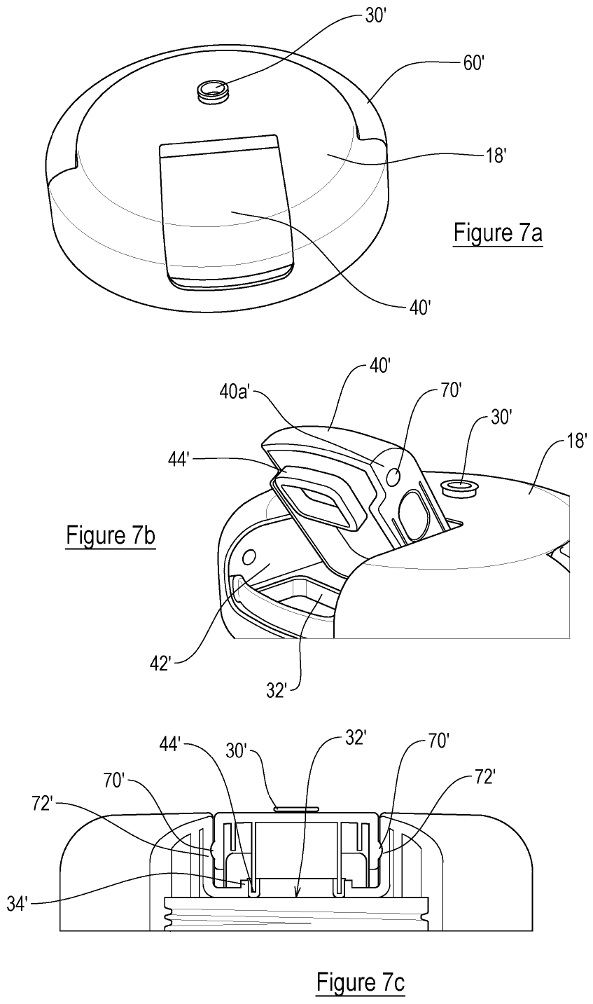

FIGS. 7a, 7b and 7c are various views of an embodiment of a top of an ostomy collection device,

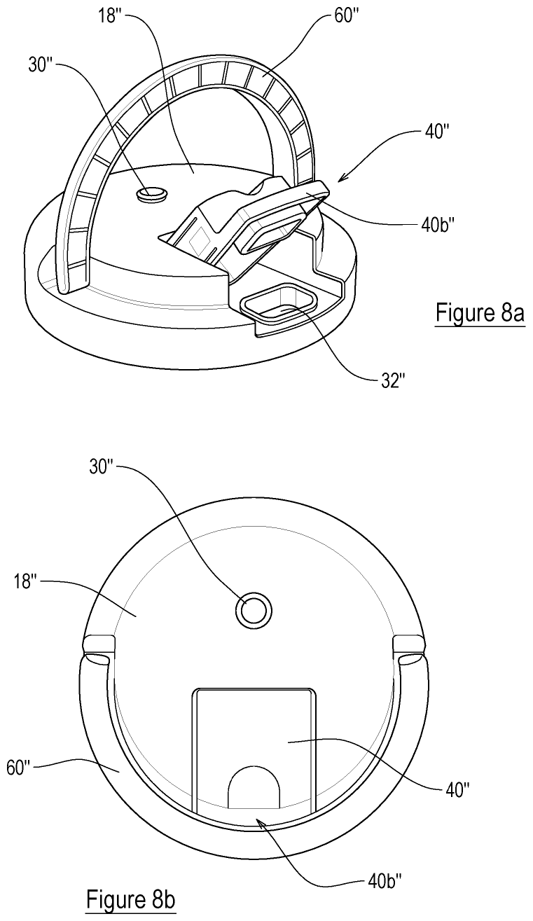

FIGS. 8a and 8b illustrate an embodiment of a top in an open and closed condition,

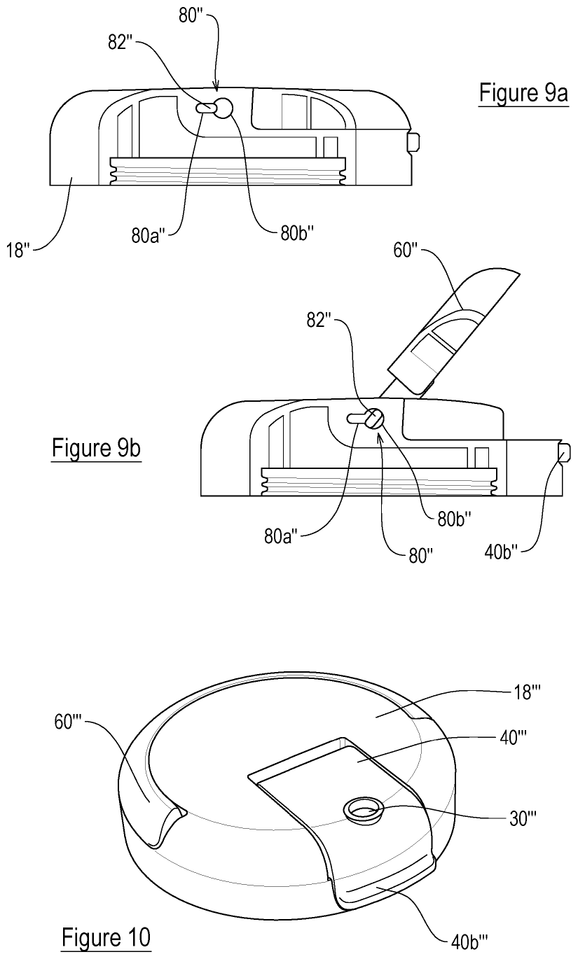

FIGS. 9a and 9b are cross-sectional views of a top in accordance with an embodiment of the invention, and

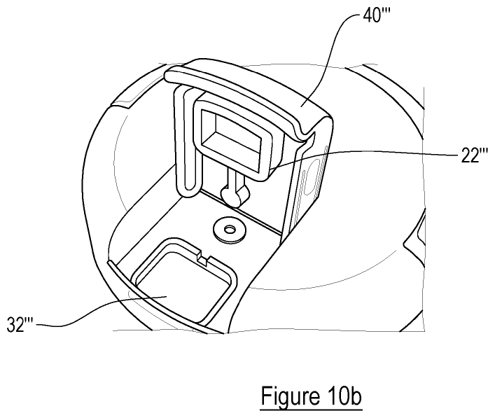

FIGS. 10a and 10b illustrate features of an embodiment of a top.

Features of an ostomy collection device 10 are shown in the figures. With particular reference to FIGS. 1 and 2, the ostomy collection device 10 is configured to connect to an outlet of a user's ostomy appliance that is connected about their stoma (and collecting waste exiting the stoma). The ostomy collection device 10 is particularly suited to urostomy use where the waste being collected is more fluid that other ostomy waste outputs and can, therefore, be emptied easily from the collection vessel into a toilet or waste disposal.

The ostomy collection device 10 includes a housing 12, which defines a collecting volume for receiving and storing waste from an ostomy appliance. The ostomy collection device 10 also includes an inlet 30, an outlet 32 and a lid portion 40.

The inlet 30 is configured to connect to an outlet of an ostomy appliance (not shown), so that waste from the ostomy appliance flows into the collecting volume. The outlet 32 is configured for emptying the contents of the collecting volume (e.g. into a toilet, etc. so that the waste can be disposed of).

The lid portion 40 is pivotable between a first position and a second position. In the first position the outlet 32 is open and waste can flow through the outlet 32 from the collecting volume. In the second position, waste flow through the outlet is impeded or substantially prevented (i.e. the outlet 32 is substantially closed).

The lid portion 40 pivots about an axis positioned at one end of the lid portion and spaced from the outlet 32, which is closer to a periphery of the top 18.

The housing 12 has a base 14, a wall 16 and a top 18. In embodiments, the base 14 provides a flat bottom portion to the ostomy collection device 10. The base 14 may be substantially circular (see figures) but it should be appreciated that this is not necessarily the case.

In embodiments, the wall 16 is connected to the base 14 (i.e. the wall 16 is connected around a peripheral edge of the base 14). The wall 16 extends substantially upwards from the base 14. In the illustrated example, there is a single continuous wall 16, which extends around the entire periphery of the base 14 to create an internal volume defined by the base 14 and the wall 16.

It should be appreciated that the wall could be constructed from multiple portions connected together, if desired.

In some embodiments, the wall 16 is ridged or concertinaed which allows the wall 16 to fold upon itself. This results in a collection device 10 that can be collapsed into a small configuration (for example, for travel).

The top 18 of the housing 12 closes the collecting volume. In the illustrated embodiments, the inlet 30 and the outlet 32 are both positioned on the top 18.

The outlet 32 is located in a recess 42 formed in the top 18 and the lid portion 40 is receivable in the recess. Thus, the lid portion 40 moves between its second position, in which the outlet 32 is closed and the lid portion 40 is received in the recess 42, and its first position, in which the outlet 32 is open and the lid portion 40 is pivoted at one end so that only a portion of the lid portion remains in the recess 42 (i.e. the part of the lid portion 40 near the pivot point/hinge 36 will stay in the recess 42, while the remainder of the lid portion 40 lifts out of the recess and sits upright extending out of the recess 42).

In some embodiments, the lid portion 40 is removable from the recess 42 (see FIG. 6, for example). In other words, the hinge 36 between the lid portion 40 and the top 18 is disconnectable, so that the lid portion 40 can be removed from the top 18. This is advantageous for cleaning purposes because the lid portion 40 can be removed which allows the top 18 and the lid portion 40 to be cleaned more easily separately. The hinge 36 may include a contoured channel 36a into which a projection 36b from the lid portion 40 is inserted, which allows the hinge 36 to provide a snap fit.

The lid portion 40 and top 18 may also have one or more dimple/recesses and bump projections that cooperate when the hinge is connected and the lid portion 40 is being used. This way multiple “stable” positions of the lid portion 40 can be provided. The first dimple recess may be at around 60 degrees, the second recess at around 75 degrees and the third at around 90 degrees—thus providing three stable states in which the lid portion 40 can be held open.

The ostomy collection device 10 also includes a locking mechanism which inhibits and/or restricts movement of the lid portion 40 out of its second position. In other words, the locking mechanism holds the lid portion 40 closed and prevent the lid portion 40 from opening (thus, revealing the outlet 32) accidently.

In some embodiments, the locking mechanism includes a first formation and a corresponding formation. The first and corresponding formations, engage each other to restrict and/or inhibit the lid portion 40 moving out of its second condition.

In some embodiments, one of the first formation or the corresponding formation includes a projection which extends outwardly from a surface. The other of the first formation and the corresponding formation includes a slot or recess. In the example illustrated in FIG. 2, the top 18 includes the projection 46 (and it extends from a mouth of the recess 42). A recess (indicated at 48) is provided on the lid 40. When the lid portion 40 is moved the its closed position, the projection 46 and the recess 48 engage to hold the lid portion 40 closed. It should be appreciated that the first and corresponding formation do not need to be arranged in the manner—further examples/embodiments are described in detail later.

Preferably, the top 18 can be removed/disconnected from the wall 16. Thus, in some embodiments, the top 18 and the wall 16 both include respective engagement formations 20a, 20b which engage with each other so that the top 18 connects to the wall 16. The engagement formations 20a, 20b are shown in detail on FIG. 8, which illustrates a cross-section view of the top 18 and the wall 16.

In some embodiments, the engagement formations 20a, 20b are corresponding threaded portions. In other words, the top 18 has an engagement formation 20a which incorporates a helical thread and the wall 16 also has an engagement formation 20b which may also be a helical thread. On relative rotation of the two helical threads together, the top 18 and the wall 16 are connected together.

In some embodiments, a seal is provided on one of the engagement formations 20a, 20b. The seal 22 may be provided as a separate part and connected to the engagement formation 20b after the wall 16/engagement formation 20b is manufactured—this is illustrated in FIG. 3. Alternatively, the seal 22 may be integral with the wall 16 (and may be moulded and formed at the same time as the rest of the wall 16)— illustrated in FIG. 4. In this example, the wall 16 extends behind the engagement formation 20b and over the top of the engagement formation 20b to form the sealing part.

In some embodiments, the housing further includes an air vent arrangement 50. The air vent arrangement 50 functions to permit gases to escape the collecting volume—this prevents or, at least, reduces pressure build up in the collecting volume that could result in malfunction.

The air vent opening 50 is illustrated in FIGS. 5a and 5b. As can be seen in FIG. 5b, the air vent arrangement 50 includes a lower part 52, which is in communication with the collecting volume and an upper part 54 which communicates through an opening 56 in the top 18 to atmosphere.

The air vent arrangement 50 includes a membrane which is water phobic to prevent/inhibit waste exiting the collecting volume through the air vent opening 56. The membrane or another part of the air vent arrangement 50 may include a carbon component, which is used to neutralise smells in the gas flowing through the air vent arrangement 50 to atmosphere.

In some embodiments, the ostomy collection device 10 further includes a user graspable handle 60. The handle 60 is pivotable between a first position (shown in FIG. 1), which is a “stowed”/storage positon, and a second position (shown in FIG. 2). In the first position in this example, the top 18 includes a second recess 62 which receives the handle 60 when it is in the first position. The handle 60, therefore, sits flush with the top surface of the ostomy collection device 10 (i.e. the handle 60 lies substantially flat or in line with the housing).

In its second position, the handle 60 is moved out of alignment with the top 18 to a useable position (i.e. position in which the user can carry the device 10 by the handle 60). The shape of the top 18 may be selected so that a “stop” formation is provided, so that the handle 60 can be positioned upright (away from the top 18) and be supported there stably.

The handle 60 is arranged between the inlet 30 and the outlet 32 (the inlet 30 and outlet 32 in this illustrated example are located on opposing sides of the top 18). The handle 60 includes a cut out portion 64, which receives (or surrounds) at least a portion of the inlet 30 (the inlet 30 in FIGS. 1 and 2 is shown with a tube connector attached).

In some embodiments, the lid portion 40 includes a flange 40b or other projection for aiding the user to move the lid portion 40.

A second embodiment of the top of the ostomy collection device is shown in FIGS. 7a to 7c. The reference numbers that are used to describe the same or similar features in this embodiment are the same as those used above with the addition of a prime symbol (i.e. the top 18 will become the top 18′). It should be appreciated that the features of the ostomy collection device 10′ not described explicitly here may be the same as those already described (for example the remainder of the housing and how it connects to the top 18′ may be identical to that described above and only some features of the top 18′ may be different). It should also be appreciated that features of this embodiment may be combined with features of another embodiment unless otherwise discussed.

In this example, the top 18′ has a different layout. The inlet 30′ is adjacent the lid portion 40′. The handle 60′ is positioned at the rear of the device (i.e. on an opposing side to the lid portion 40′) and extends around the periphery of the top 18′ (similarly to the example illustrated in FIGS. 1 and 2). However, there is no cut out in the handle 60′ for the inlet 30′. The inlet 30′ is in a more central position on the top 18′.

In some embodiments (such as the example in FIGS. 7a to 7c), the first formation of the locking mechanism includes a projection 70′ that extends “sideways” of the lid portion 40′. In other words, the projection 70′ extends outwards from (an outwardly facing side surface 40a′ of) the lid portion 40′ and generally parallel to the pivot axis about which the lid portion 40′ pivots to move between the first and second positions (open and closed positions).

The corresponding formation of the locking mechanism includes a recess 72′. The recess 72′ is formed in an inwardly facing surface of the top 18′ and engages the projection 70′ when the lid portion 40′ is moved to its closed position (shown in the cut away FIG. 7c). Thus, the locking mechanism holds the lid portion 40′ closed and prevents it opening accidently or inadvertently (for example, if the ostomy collection device is knocked over).

In the example illustrated in FIGS. 7a to 7c, the first formation of the locking mechanism includes a pair of projections 70′, one of the pair is positioned on opposing outwardly facing side surfaces of the lid portion 40′. It follows on that the corresponding formation includes two recesses 72′, each positioned on opposing inwardly facing surfaces of the top 18′ (i.e. the vertical surfaces of the recess 42′).

In some embodiments, the lid portion 40′ includes a seal portion 44′. The seal portion 44′ is configured to extend around the outlet 32′ when the lid portion 40′ is in its second position. The top 18′ includes a rim or ridge 34′ which defines the outlet 32 opening. The seal portion 44′ contacts the rim 34′ to form the seal and prevents liquid from flowing out of the outlet 32′. The seal portion 44′ may have a friction fit against the top 18′ (i.e. the rim 44′), which also aids the resistance of the lid portion 40′ being moved from the second, closed, position.

A third embodiment of the top of the ostomy collection device is shown in FIGS. 8a to 9b. The reference numbers that are used to describe the same or similar features in this embodiment are the same as those used above with the addition of a double prime symbol (i.e. the top 18 will become the top 18″). It should be appreciated that the features of the ostomy collection device 10″ not described explicitly here may be the same as those already described (for example the remainder of the housing and how it connects to the top 18″ may be identical to that described above and only some features of the top 18″ may be different). It should also be appreciated that features of this embodiment may be combined with features of another embodiment unless otherwise discussed.

In this example, the first formation and the corresponding formation of the locking mechanism are moveable translationally relative to each other. In other words, the first formation or the corresponding formation is movable translationally in the other of the first formation and the corresponding formation. The translational movement allows the first and corresponding formation to move between a locking position and a rotating position. In the locking position, the lid portion 40″ is inhibited and/or prevented from rotating. In other words, the lid portion 40″ is held in a position in which rotation is not possible. In this example, the lid portion 44″ is held in its second position, so that the outlet 32″ is closed.

In the rotating position, the first formation and the corresponding formation form a hinge, which allows a part to rotate. In this example, the first formation and corresponding formation are provided by the handle 60″ and the top 18″. Two recesses 80″ are provided by the top 18″ or the handle 60″ and a pair of projections 82″ are provided by the other of the top 18″ and handle 60″ (or alternatively, one recess may be on the top 18″ and one may be on the handle 60″ and each of the handle 60″ and the top 18″ also includes one of the projections 82″). It should be appreciated that the recess(es) and projection(s) could also be provided on the top 18″ and the top portion 40″.

Each recess 80″ includes a narrowed channel part 80a″ and a wider part 80b″ and the projection 82″ is received in the recess 80″. When the locking mechanism is in the locking position, the projection(s) 82″ is positioned in the narrowed channel part 80a″ (see, for example, FIG. 80a). In other words, to move to the locking position, the projection 82″ is moved along the recess 80″ and into the narrowed channel part 80a″, which part 80a″ prevents rotation of the projection 82″.

When the locking mechanism is in the rotating position, the projection 82″ is positioned in the wider part 80b″. The projection 82″ is permitted to rotate with the wider part 80b″ and as such the locking mechanism is in the rotating positon (see, for example, FIG. 9b). In this example, the wider part 80b″ is generally circular but it should be appreciated that this is not necessarily the case.

In the illustrated example, the locking mechanism is positioned on part of the handle 60″ and its interaction with the top 18″. The handle 60″ holds the lid portion 40″ in its second position by virtue of the contact between a flange part of the lid portion 40″ and the handle 60″. This is shown clearest in FIG. 7b, where the flange part 40b″ is pinned under the handle 60″. Thus, when the handle 60″ is locked and cannot rotate, the lid portion 40″ is also held in the closed position and not able to move away from the second position.

When the handle 60″ is moved to the rotating position and rotated upwards (away from the top surface of the top 18″), then the lid portion 40″ is free to move out of its second position (e.g. the lid portion 40″ can be pivoted away from the second position and towards its first position).

In the above example, the hinge for the handle 60″ is provided by the first and corresponding formations. This may be preferable since the hinge 36 between the lid portion 40″ and the top 18″ can then be disconnectable (as discussed above) at the same time hinge between the handle 60″ and top 18″ providing the locking mechanism.

However, it should be appreciated that the hinge for the lid portion 40″/top 18″ may be provided by the first and corresponding formations of the locking mechanism.

A fourth embodiment of the top of the ostomy collection device is shown in FIGS. 10a and 10b. The reference numbers that are used to describe the same or similar features in this embodiment are the same as those used above with the addition of a double prime symbol (i.e. the top 18 will become the top 18″). It should be appreciated that the features of the ostomy collection device 10 not described explicitly here may be the same as those already described (for example the remainder of the housing and how it connects to the top 18″ may be identical to that described above and only some features of the top 18″ may be different). It should also be appreciated that features of this embodiment may be combined with features of another embodiment unless otherwise discussed.

In some embodiments, the inlet 30″ is positioned on the lid portion 40″. When the lid portion 40″ is in its second (closed) position, the inlet 30″ aligns with the outlet 32″ (which is under the lid portion 40″). In other words, when the lid portion 40″ is in its closed position, the inlet 30″ is in fluid communication with the outlet 32″ and the collecting volume with in the ostomy collection device.

In the second position of the lid portion 40′″, waste flow is impeded because the inlet 30 is aligned with a portion of the outlet 32, which means the outlet 32 is not “completely” closed. Although not closed, when the device 10″ is in use, the inlet 30″ will be connected to a delivery tube which is connected to the ostomy appliance of the user—thus, even if the ostomy collection device 10 is knocked over, the contents will not spill easily unless the outlet 32 is properly opened by the lid portion 40.

In this case it may be preferable that the inlet 30″ includes a one way valve. The valve may be opened by the connector from the tube of an ostomy appliance and otherwise biased to a closed condition. Thus, irrespective of where the inlet 30″ is positioned, unless that connector is connected to the inlet 30″, the inlet 30″ is closed. This means that even if the inlet 30″ is positioned on the lid portion 40″, the second position of the lid portion 40″ closes the outlet 32″.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy collection device including:
a housing which defines a collecting volume for receiving and storing waste from an ostomy appliance,
an inlet for fluidly connecting to an outlet of an ostomy appliance so that waste from the ostomy appliance flows into the collecting volume,
an outlet for emptying the contents of the collecting volume, and
a lid portion which is pivotable between a first position, in which the outlet is open and waste can flow through the outlet from the collecting volume, and a second position in which waste flow through the outlet is impeded or substantially prevented.

2. An ostomy collection device according to claim 1 wherein the housing includes a top and the inlet and the outlet are positioned in or on the top.

3. An ostomy collection device according to claim 2 wherein the outlet is located in a recess formed in the top and the lid portion is receivable in the recess.

4. An ostomy collection device according to claim 1 further including a locking mechanism which inhibits and/or restricts movement of the lid portion out of its second position.

5. An ostomy collection device according to claim 4 wherein the locking mechanism includes a first formation and a corresponding formation which are engageable with each other to restrict and/or inhibit the lid portion moving out of its second condition.

6. An ostomy device according to claim 5 wherein the first formation or corresponding formation includes a projection which extends outwardly from a surface and the other of the first formation and the corresponding formation includes a slot or recess.

7. An ostomy collection device according to claim 6 wherein the first formation is located on the lid portion and the corresponding formation is on or in the housing.

8. An ostomy collection device according to claim 4 wherein the first formation or the corresponding formation is movable translationally in the other of the first formation and the corresponding formation between a locking position in which the lid portion is inhibited and/or prevented from rotating and a rotating position in which the first formation and the corresponding formation form a hinge.

9. An ostomy collection device according to claim 8 wherein the locking mechanism further includes a member which is attached to the first formation and moves between the locking position in which it holds the lid portion closed and rotating position in which the member is operable to move away from the lid portion and allow the lid portion to move to its open position.

10. An ostomy collection device according to claim 8 wherein the first formation or the corresponding formation includes a narrowed channel and a wider rotation opening.

11. An ostomy collection device according to claim 2 wherein the housing includes a base and a wall and the top is connectable to and disconnectable from the wall.

12. An ostomy collection device according to claim 11 wherein the top and the wall include respective engagement formations which are engageable to seal the top to the wall.

13. An ostomy collection device according to claim 12 wherein the engagement formations are corresponding helical threaded portions.

14. An ostomy collection device according to claim 12 wherein a seal is provide on one of the engagement formations on the wall or the top, and is optionally formed integrally with the respective engagement formation.

15. An ostomy collection device according to claim 1 wherein the housing further includes an air vent arrangement.

16. An ostomy collection device according to claim 15 wherein the air vent arrangement includes a membrane which is water phobic to prevent or inhibit waste exiting the collecting volume through the air vent arrangement.

17. An ostomy collection device according to claim 15 wherein the housing includes a top and the inlet and the outlet are positioned in or on the top and wherein the air vent arrangement includes an opening in the top.

18. An ostomy collection device according to claim 1 wherein when the lid portion is in the second position the outlet is closed and optionally, sealed closed.

19. An ostomy collection device according to claim 1 further including a user graspable handle, and optionally the user graspable handle is pivotable between a first position and a second position.

20. An ostomy collection device according to claim 19 wherein the first handle position is a carrying position in which the handle is substantially upright and the second handle position is a stowed position in which the handle lies substantially flat or in line with the housing.

21. An ostomy collection device according to claim 20 wherein the housing includes a top and the inlet and the outlet are positioned in or on the top and wherein the top includes a recess for receiving the handle in the stowed position.

22. An ostomy collection device according to claim 1 wherein the lid portion includes a projection for aiding the user to move the lid portion.

23. An ostomy collection device according to claim 2 wherein the inlet is located on an opposing side of the top to the outlet.

24. An ostomy collection device according to claim 22 wherein the user graspable handle is positioned between the inlet and the outlet.

25. An ostomy collection device according to claim 1 wherein the inlet is located on the lid portion and when the lid portion is in its second position the inlet is in fluid communication with the outlet and the collecting volume.
